(19) United States
(12) Patent Application Publication
(10) Pub. No.: US 2024/0347181 A1
(43) Pub. Date: Oct. 17, 2024

(54) SYSTEM AND METHOD FOR FACILITATING SHORT-TERM STAFFING IN THE HEALTHCARE INDUSTRY

(71) Applicant: Jeremy Jones, Cedar Park, TX (US)

(72) Inventor: Jeremy Jones, Cedar Park, TX (US)

(21) Appl. No.: 18/582,275

(22) Filed: Feb. 20, 2024

Related U.S. Application Data
(63) Continuation-in-part of application No. 16/679,419, filed on Nov. 11, 2019, now abandoned.

(51) Int. Cl. G16H 40/20; G06Q 10/0631; G06Q 10/1053

(52) U.S. Cl. CPC G16H 40/20; G06Q 10/063112; G06Q 10/1053

(57) ABSTRACT

A system for obtaining staffing for a plurality of temporary staffing opportunities available at various healthcare facilities is disclosed. The system comprises a computing device in operable communication with a database to store information related to a plurality of candidates and a plurality of healthcare facilities. A calendar module receives scheduling information via the computing device and corresponds the scheduling information to a plurality of staffing availabilities. A hiring system transmits the plurality of staffing availabilities to the plurality of candidates, and a comparator compares data pertaining to the plurality of candidates with the plurality of staffing availabilities to match one or more of the plurality of candidates with one or more of the plurality of staffing availabilities.

### CROSS-REFERENCE TO RELATED APPLICATIONS

The present application is a Continuation-In-Part and claims priority to U.S. Non-Provisional application Ser. No. 16/679,419 filed Nov. 11, 2019, titled “SYSTEM AND METHOD FOR FACILITATING SHORT-TERM STAFFING IN THE HEALTHCARE INDUSTRY,” which is hereby incorporated by reference in its entirety.

### TECHNICAL FIELD

The embodiments generally relate to electronic services and more specifically relate to a system for facilitating staffing of a labor force via a third-party job placement service.

### BACKGROUND

The healthcare industry often suffers from understaffing, causing a deficit in the labor force. It can prove difficult for healthcare providers to source and adequately recruit quality candidates for various job openings. The American Hospital Association projects that the rate of understaffing will continue to rise in the coming years.

The growing demand for qualified healthcare professionals in the travel nurse industry requires healthcare facilities to rely on travel nurse agencies to source and staff open positions from a limited pool of healthcare professionals. To compound the problem, many of the healthcare professionals are seeking open positions in a specialized field of the healthcare industry.

There are currently no convenient resources for facilitating the recruitment and staffing of travel healthcare professions (such as travel nurses). This results in the personnel at the healthcare facility being forced to source and staff travel healthcare professionals using various staffing agencies, online resources, or by vetting applicants in-house. This additional effort results in a loss of time and economic resources, which in turn causes a multitude of downstream problems throughout the healthcare industry.

### SUMMARY OF THE INVENTION

This summary is provided to introduce a variety of concepts in a simplified form that is further disclosed in the detailed description of the embodiments. This summary is not intended to identify key or essential inventive concepts of the claimed subject matter, nor is it intended for determining the scope of the claimed subject matter.

The embodiments disclosed herein relate to a system for obtaining staffing for a plurality of temporary staffing opportunities available at various healthcare facilities. The system comprises a computing device in operable communication with a database to store information related to a plurality of candidates and a plurality of healthcare facilities. A calendar module receives scheduling information via the computing device and corresponds the scheduling information to a plurality of staffing availabilities. A hiring system transmits the plurality of staffing availabilities to the plurality of candidates, and a comparator compares data pertaining to the plurality of candidates with the plurality of staffing availabilities to match one or more of the plurality of candidates with one or more of the plurality of staffing availabilities.

In one aspect, a revenue prediction module provides revenue forecasting related to each of the plurality of candidates.

In one aspect, an evaluation module receives a plurality of recruiter data to generate a recruiter score using the plurality of data.

In one aspect, an evaluation module is configured to receive one or more evaluations and correspond the evaluation to one of the plurality of healthcare professionals.

In one aspect, a plurality of candidate activity data is transmitted to the revenue prediction module to identify revenue-generating candidates.

In one aspect, the evaluation module provides a recruiter score optimization to optimize future revenues.

In one aspect, one or more revenue calculation predictions are provided based on the plurality of candidate activity data.

In one aspect, a verification module verifies one or more credentials provided by the plurality of candidates. The one or more credentials are comprised of one or more of the following: a vaccination history, one or more licenses, and one or more qualifications.

In one aspect, an employment history is stored in the database which may be viewed by personnel at the healthcare facility to determine a suitable hirable candidate.

In one aspect, an evaluation module receives one or more evaluations and correspond the evaluation to one of the plurality of healthcare professionals. Evaluations may be provided by a healthcare facility, by candidates, or by other users of the system.

In one aspect, a calendar module indicates one or more employable candidates to a user at the healthcare facility.

In one aspect, the scheduling information comprises one or more of the following: one or more dates available, one or more days of the week available, one or more locations, a travel distance, and one or more times available.

In one aspect, a communication module provides a communication means between the plurality of healthcare facilities and the plurality of candidates.

### BRIEF DESCRIPTION OF THE DRAWINGS

A complete understanding of the present embodiments and the advantages and features thereof will be more readily understood by reference to the following detailed description when considered in conjunction with the accompanying drawings wherein:

FIG. 1 illustrates a block diagram of the network infrastructure, according to some embodiments;

FIG. 2 illustrates a block diagram of the network infrastructure, according to some embodiments;

FIG. 3 illustrates a block diagram of the server engine and associated modules, according to some embodiments;

FIG. 4 illustrates a flowchart of a use case diagram, according to some embodiments;

FIG. 5 illustrates a block diagram of a candidate credential table, according to some embodiments;

FIG. 6 illustrates a view of the offer generation system, according to some embodiments; and

FIG. 7 illustrates a flowchart of a method for staffing temporary position, according to some embodiments.

### DETAILED DESCRIPTION

The specific details of the single embodiment or variety of embodiments described herein are to the described system and methods of use. Any specific details of the embodiments are used for demonstrative purposes only, and no unnecessary limitations or inferences are to be understood therefrom.

Before describing in detail exemplary embodiments, it is noted that the embodiments reside primarily in combinations of components and procedures related to the system. Accordingly, the system components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

In general, the embodiments described herein provide systems and methods for registering, staffing, and evaluating a workforce for a facility. The embodiments may be used to recruit candidates, skilled laborers, and/or unskilled laborers to fill temporary and/or permanent positions available at a facility, such as a healthcare facility. More specifically, candidates may be provided with access to more employment opportunities in various locations during pre-specified periods of time. The system may allow for performance-based pay and control over a work schedule. At the same time, hiring facilities, such as healthcare facilities may gain the benefit of accessing a larger pool of potential candidates from which they may review candidate credentials, candidate schedules, and contact the candidate. As a result, the system greatly improves hiring efficiency while improving the overall candidate quality.

As used herein, the term “user(s)” may refer to candidates who are seeking staffing opportunities at various facilities, hiring managers at the facilities, representatives of the facilities, administrative users, or other professionals involved in the hiring process. In a particular example, the candidate is a healthcare professional (such as a nurse, travel nurse, or other profession in the healthcare setting). The system may be particularly useful to healthcare professionals who frequently travel to various locations and are employed at each location temporarily, such as travel nurses. To participate in the staffing marketplace, users may register with the system. The registration process may involve several steps, and in some embodiments, may involve taking screening tests, answering surveys, providing references, confirming employment and/or education history, confirming licensure status, providing documents, and/or executing contracts. Some of the registration steps may be performed using the system, and some may be performed manually using paper documents, mail and/or in person interviews.

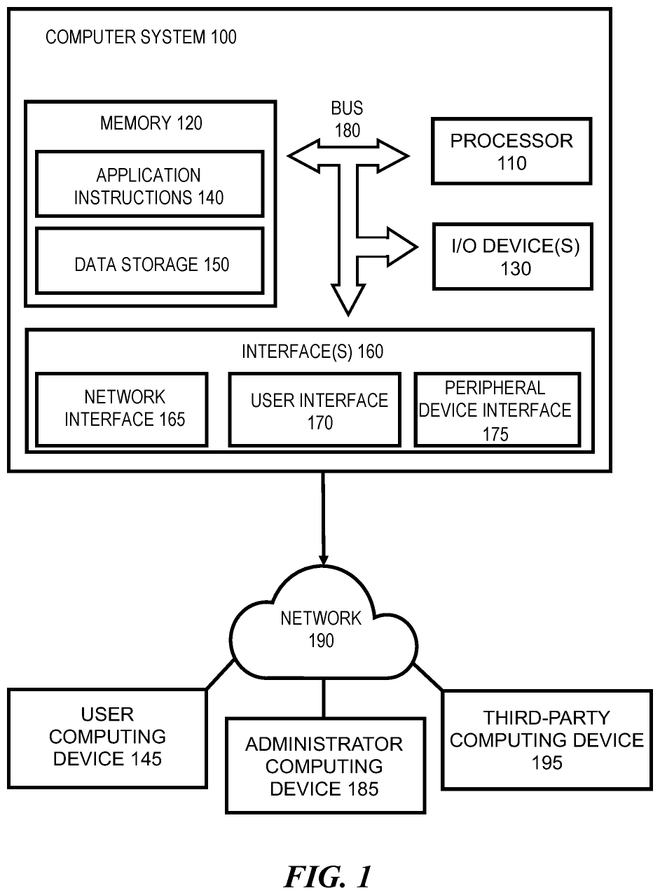

FIG. 1 illustrates an example of a computer system 100 that may be utilized to execute various procedures, including the processes described herein. The computer system 100 comprises a standalone computer or mobile computing device, a mainframe computer system, a workstation, a network computer, a desktop computer, a laptop, or the like. The computing device 100 can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive).

In some embodiments, the computer system 100 includes one or more processors 110 coupled to a memory 120 through a system bus 180 that couples various system components, such as an input/output (I/O) devices 130, to the processors 110. The bus 180 may be any of several types of bus structures including a memory bus or memory controller, a peripheral bus, and a local bus using any of a variety of bus architectures. For example, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus, also known as Mezzanine bus.

In some embodiments, the computer system 100 includes one or more input/output (I/O) devices 130, such as video device(s) (e.g., a camera), audio device(s), and display(s) are in operable communication with the computer system 100. In some embodiments, similar I/O devices 130 may be separate from the computer system 100 and may interact with one or more nodes of the computer system 100 through a wired or wireless connection, such as over a network interface.

Processors 110 suitable for the execution of computer readable program instructions include both general and special purpose microprocessors and any one or more processors of any digital computing device. For example, each processor 110 may be a single processing unit or a number of processing units and may include single or multiple computing units or multiple processing cores. The processor(s) 110 can be implemented as one or more microprocessors, microcomputers, microcontrollers, digital signal processors, central processing units, state machines, logic circuitries, and/or any devices that manipulate signals based on operational instructions. For example, the processor(s) 110 may be one or more hardware processors and/or logic circuits of any suitable type specifically programmed or configured to execute the algorithms and processes described herein. The processor(s) 110 can be configured to fetch and execute computer readable program instructions stored in the computer-readable media, which can program the processor(s) 110 to perform the functions described herein.

In this disclosure, the term “processor” can refer to substantially any computing processing unit or device, including single-core processors, single-processors with software multithreading execution capability, multi-core processors, multi-core processors with software multithreading execution capability, multi-core processors with hardware multithread technology, parallel platforms, and parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. Further, processors can exploit nano-scale architectures, such as molecular and quantum-dot based transistors, switches, and gates, to optimize space usage or enhance performance of user equipment. A processor can also be implemented as a combination of computing processing units.

In some embodiments, the memory 120 includes computer-readable application instructions 150, configured to implement certain embodiments described herein, and a database 150, comprising various data accessible by the application instructions 140. In some embodiments, the application instructions 140 include software elements corresponding to one or more of the various embodiments described herein. For example, application instructions 140 may be implemented in various embodiments using any desired programming language, scripting language, or combination of programming and/or scripting languages (e.g., Android, C, C++, C#, JAVA, JAVASCRIPT, PERL, etc.).

In this disclosure, terms “store,” “storage,” “data store,” data storage,” “database,” and substantially any other information storage component relevant to operation and functionality of a component are utilized to refer to “memory components,” which are entities embodied in a “memory,” or components comprising a memory. Those skilled in the art would appreciate that the memory and/or memory components described herein can be volatile memory, nonvolatile memory, or both volatile and nonvolatile memory. Nonvolatile memory can include, for example, read only memory (ROM), programmable ROM (PROM), electrically programmable ROM (EPROM), electrically erasable ROM (EEPROM), flash memory, or nonvolatile random access memory (RAM) (e.g., ferroelectric RAM (FeRAM). Volatile memory can include, for example, RAM, which can act as external cache memory. The memory and/or memory components of the systems or computer-implemented methods can include the foregoing or other suitable types of memory.

Generally, a computing device will also include, or be operatively coupled to receive data from or transfer data to, or both, one or more mass data storage devices; however, a computing device need not have such devices. The computer readable storage medium (or media) can be a tangible device that can retain and store instructions for use by an instruction execution device. The computer readable storage medium can be, for example, an electronic storage device, a magnetic storage device, an optical storage device, an electromagnetic storage device, a semiconductor storage device, or any suitable combination of the foregoing. A non-exhaustive list of more specific examples of the computer readable storage medium can include: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. In this disclosure, a computer readable storage medium is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

In some embodiments, the steps and actions of the application instructions 140 described herein are embodied directly in hardware, in a software module executed by a processor, or in a combination of the two. A software module may reside in RAM, flash memory, ROM memory, EPROM memory, EEPROM memory, registers, a hard disk, a removable disk, a CD-ROM, or any other form of storage medium known in the art. An exemplary storage medium may be coupled to the processor 110 such that the processor 110 can read information from, and write information to, the storage medium. In the alternative, the storage medium may be integrated into the processor 110. Further, in some embodiments, the processor 110 and the storage medium may reside in an Application Specific Integrated Circuit (ASIC). In the alternative, the processor and the storage medium may reside as discrete components in a computing device. Additionally, in some embodiments, the events or actions of a method or algorithm may reside as one or any combination or set of codes and instructions on a machine-readable medium or computer-readable medium, which may be incorporated into a computer program product.

In some embodiments, the application instructions 140 for carrying out operations of the present disclosure can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The application instructions 140 can execute entirely on the user's computer, partly on the user's computer, as a stand-alone software package, partly on the user's computer and partly on a remote computer, or entirely on the remote computer or server. In the latter scenario, the remote computer can be connected to the user's computer through any type of network, including a local area network (LAN) or a wide area network (WAN), or the connection can be made to an external computer (for example, through the Internet using an Internet Service Provider). In some embodiments, electronic circuitry including, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform aspects of the present disclosure.

In some embodiments, the application instructions 140 can be downloaded to a computing/processing device from a computer readable storage medium, or to an external computer or external storage device via a network 190. A network adapter card or network interface in each computing/processing device receives computer readable program instructions from the network and forwards the computer readable application instructions 140 for storage in a computer readable storage medium within the respective computing/processing device.

In some embodiments, the computer system 100 includes one or more interfaces 160 that allow the computer system 100 to interact with other systems, devices, or computing environments. In some embodiments, the computer system 100 comprises a network interface 165 to communicate with a network 190. In some embodiments, the network interface 165 is configured to allow data to be exchanged between the computer system 100 and other devices attached to the network 190, such as other computer systems, or between nodes of the computer system 100. In various embodiments, the network interface 165 may support communication via wired or wireless general data networks, such as any suitable type of Ethernet network, for example, via telecommunications/telephony networks such as analog voice networks or digital fiber communications networks, via storage area networks such as Fiber Channel SANs, or via any other suitable type of network and/or protocol. Other interfaces include the user interface 170 and the peripheral device interface 175.

In some embodiments, the network 190 corresponds to a local area network (LAN), wide area network (WAN), the Internet, a direct peer-to-peer network (e.g., device to device Wi-Fi, Bluetooth, etc.), and/or an indirect peer-to-peer network (e.g., devices communicating through a server, router, or other network device). The network 190 can comprise copper transmission cables, optical transmission fibers, wireless transmission, routers, firewalls, switches, gateway computers and/or edge servers. The network 190 can represent a single network or multiple networks. In some embodiments, the network 190 used by the various devices of the computer system 100 is selected based on the proximity of the devices to one another or some other factor. For example, when a first user device and second user device are near each other (e.g., within a threshold distance, within direct communication range, etc.), the first user device may exchange data using a direct peer-to-peer network. But when the first user device and the second user device are not near each other, the first user device and the second user device may exchange data using a peer-to-peer network (e.g., the Internet). The Internet refers to the specific collection of networks and routers communicating using an Internet Protocol (“IP”) including higher level protocols, such as Transmission Control Protocol/Internet Protocol (“TCP/IP”) or the Uniform Datagram Packet/Internet Protocol (“UDP/IP”).

Any connection between the components of the system may be associated with a computer-readable medium. For example, if software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. As used herein, the terms “disk” and “disc” include compact disc (CD), laser disc, optical disc, digital versatile disc (DVD), floppy disk, and Blu-ray disc; in which “disks” usually reproduce data magnetically, and “discs” usually reproduce data optically with lasers. Combinations of the above should also be included within the scope of computer-readable media. In some embodiments, the computer-readable media includes volatile and nonvolatile memory and/or removable and non-removable media implemented in any type of technology for storage of information, such as computer-readable instructions, data structures, program modules, or other data. Such computer-readable media may include RAM, ROM, EEPROM, flash memory or other memory technology, optical storage, solid state storage, magnetic tape, magnetic disk storage, RAID storage systems, storage arrays, network attached storage, storage area networks, cloud storage, or any other medium that can be used to store the desired information and that can be accessed by a computing device. Depending on the configuration of the computing device, the computer-readable media may be a type of computer-readable storage media and/or a tangible non-transitory media to the extent that when mentioned, non-transitory computer-readable media exclude media such as energy, carrier signals, electromagnetic waves, and signals per se.

In some embodiments, the system is world-wide-web (www) based, and the network server is a web server delivering HTML, XML, etc., web pages to the computing devices. In other embodiments, a client-server architecture may be implemented, in which a network server executes enterprise and custom software, exchanging data with custom client applications running on the computing device.

In some embodiments, the system can also be implemented in cloud computing environments. In this context, “cloud computing” refers to a model for enabling ubiquitous, convenient, on-demand network access to a shared pool of configurable computing resources (e.g., networks, servers, storage, applications, and services) that can be rapidly provisioned via virtualization and released with minimal management effort or service provider interaction, and then scaled accordingly. A cloud model can be composed of various characteristics (e.g., on-demand self-service, broad network access, resource pooling, rapid elasticity, measured service, etc.), service models (e.g., Software as a Service (“SaaS”), Platform as a Service (“PaaS”), Infrastructure as a Service (“IaaS”), and deployment models (e.g., private cloud, community cloud, public cloud, hybrid cloud, etc.).

As used herein, the term “add-on” (or “plug-in”) refers to computing instructions configured to extend the functionality of a computer program, where the add-on is developed specifically for the computer program. The term “add-on data” refers to data included with, generated by, or organized by an add-on. Computer programs can include computing instructions, or an application programming interface (API) configured for communication between the computer program and an add-on. For example, a computer program can be configured to look in a specific directory for add-ons developed for the specific computer program. To add an add-on to a computer program, for example, a user can download the add-on from a website and install the add-on in an appropriate directory on the user's computer.

In some embodiments, the computer system 100 may include a user computing device 145, an administrator computing device 185 and a third-party computing device 195 each in communication via the network 190. The administrator computing device 185 is utilized by an administrative user to moderate content and to perform other administrative functions. The third-party computing device 195 may be utilized by third parties to receive communications from the user computing device, transmit communications to the user via the network, and otherwise interact with the various functionalities of the system.

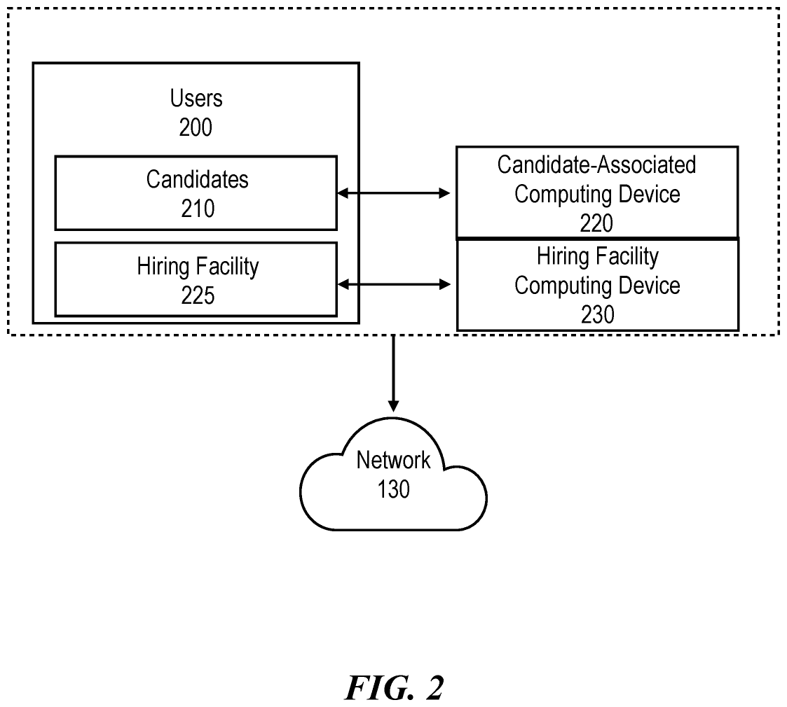

In reference to FIG. 2, a schematic of the system is disclosed, comprising a community of users 200 which include a plurality of candidates 210 in communication with a candidate-associated computing device 220, and a plurality of hiring professionals associated with a hiring facility 225 (such as a healthcare facility) in communication with a hiring facility computing device 230. Each user in the community is in communication with the system and other users via the network 130. The facility 225 may include a secure network 240 to allow secure communications to and from the facility 225 as well as communications within the facility 225.

In one embodiment, the candidate 210 may be a health care worker, such as a Nurse (e.g. Nurse Practitioner (“NP”), Registered Nurse (“RN”), Licensed Practical Nurse (“LPN”), Licensed Vocational Nurse (“LVN”), Certified Nurse's Assistant (“CNA”), and/or a Health Care Aide (“HCA”)). In such cases, the hiring facility 225 may be a health care provider such as a hospital, nursing home, or other facility, or a private residence. A hiring facility 225 may authorize an individual to act as a manager who can perform administrative tasks within the system on behalf of the hiring facility 225.

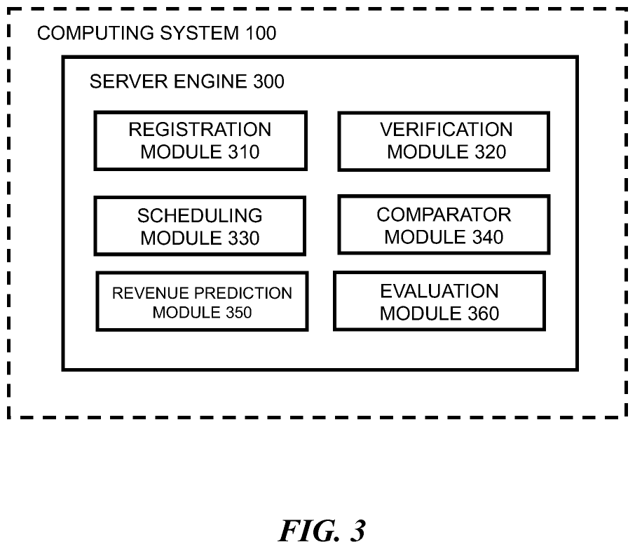

FIG. 3 illustrates a server engine 300 comprised of a plurality of modules operable to perform the plurality of tasks described by the various embodiments. A registration module 310 transmits user registration information and categorizes the information. Registration information may include user contact information, user-type (e.g., candidate, hiring personnel, facility representative, administrator, etc.), user login credentials, usernames, passwords, and other information known in the arts. The registration module 310 may also operate to collect and categorize user credentials such as work history, resumes, licenses, experience, and other credentials, especially those common in regulated skilled labor industries. A verification module 320 operates to verify credentials input by the user. Credentials may include resumes, certifications, licenses, professional experience, professional references, degrees, and other professional credentials known in the arts. Further the verification module 320 may verify a vaccine history of the user, which may be especially useful for users working in the healthcare industry. A scheduling module 330 allows the candidate to input availability information, such as dates, times, and locations, for which they are available to work, which will be matched by a comparator 340 to staffing availabilities analyzed by a calendar module which receives input from users at the facilities.

In some embodiments, credentials may include license information and/or certification information which is requested or received by the system. The information may include location and type of license/credential, when the license/certificate was issued, the time period for which the license/certificate is valid, and an expiration date thereof if applicable.

Credential information may include such items as the type of credential or skill, a subcategory of a specialized skill within the selected broad category, a short description of the skill, the number of years of experience that the worker has with that skill, and the years since the worker's most recent use of the skill (e.g., currently using this skill, in the past 12 months, in the last 3 years, over 3 years ago). Certificates information may include, for example, for a nurse, an expiration date of a nursing license, an expiration date of a CPR certification, an expiration date of a TB immunization, an expiration date of a current physical and/or the date of Hepatitis B Immunization.

In some embodiments, the availability information may include dates, days of the week, times that the worker is available or willing to work, a preferred geographic location, and/or a minimum travel distance. In some instances, the method also includes collecting registration information from the worker, such as a name, email address, phone number, licensure information, union membership information, skills, previous work history, and similar information. In some cases, the registration information may include a preferred method of communication (e.g., email, cell phone). In some implementations, the matching process may also be based on the workers' skills, skill level, number of years using the skill, and/or how recently the skill was used.

In some embodiments, the workers may be screened prior to being considered for the matching process. Screening may be performed by the verification module 320. The screening may include the administration of one or more tests aimed at gauging the worker's knowledge of a particular subject area or skill. In the travel nursing context, as just a few of the many possible examples, the tests may require the workers to identify certain drug interactions, recite the process for administering an intravenous drip, or calculate dosages.

In some embodiments, a revenue prediction module 350 provides a predictive model for future revenues for the staffing agency. The revenue prediction module 350 may utilize historical week-over-week (WoW) revenue data, known future revenue (signed contract values), recruiter scores, and candidate activity data to forecast future revenues. It will also account for the varying duration of contracts, which typically last 13 weeks but can be influenced by extensions and cancellations. The revenue prediction module 350 aims to provide a reliable and accurate prediction model that can help the agency to plan and strategize its operations based on projected revenues.

In some embodiments, an evaluation module 360 provides a means for assessing recruiters with the goal of improving predictive capabilities of recruiter scores. The evaluation module 360 may use a points-based system to determine recruiter scores, which are anticipated to have a positive correlation with revenues. The evaluation module 360 will analyze the relationship between recruiter scores and revenues, identify the key components of the score contributing to revenue generation, and suggest improvements to the scoring system.

In some embodiments, the system is operable to optimize the staffing process by incorporating candidate activity data into the staffing and revenue prediction processes. Candidate activities, ranging from completing the initial employment application, performing job searches, to requesting job submissions, are currently tracked but not weighted. This project will develop a weighted scoring system for different candidate activities, using the weighted scores to enhance both the revenue prediction model and the staffing process.

Each module detailed in FIG. 3 requires the processes of preparing and exploring the data. As a data-driven project, the success of efforts to optimize revenue prediction and staffing processes largely depends on the quality and structure of data. Therefore, thorough data preparation and exploration are crucial tasks that set the foundation for the subsequent stages of the project.

Data preparation involves gathering the required data, cleaning it, and transforming it into a format that will be suitable for analysis. Data cleaning tasks may include identifying and handling missing values, detecting and correcting errors, and dealing with outliers. Transformation processes often involve normalizing the data, handling categorical variables, and potentially creating new features that might be useful for the models.

The data preparation phase also includes consolidating data from multiple sources, ensuring consistency in the data sets, and arranging the data in a manner suitable for further analysis. The system may ensure that the data is appropriately split into training and test sets, to allow for model validation in the later stages of the project.

During data exploration processes, the system works to understand the characteristics, patterns, and relationships present in the data. Through descriptive statistics and data visualization techniques, the system aims to obtain a clear understanding of what the data is telling us, and how different variables might relate to each other. The insights gained during this phase can guide the system's selection of suitable modeling techniques and help us make informed decisions about the direction of the project.

Data sources utilized by the system include revenue data, recruiter scores data, candidate activity data, job submission data, and employer cost data. The historical revenue data source for this project is the week-over-week (WoW) candidate specific revenues. This data is collected from the payroll and current compliance system, Contingent Talent Management System (CTMS) and includes detailed information about the revenues generated each week. The future revenue data source for this project is derived from third-party sources which includes data on known revenue from signed and confirmed contracts. These data sources will be critical in training the predictive models.

Another key dataset is the historical day-over-day (DoD) recruiter scores. These scores are based on a point system that assesses the effectiveness of a recruiter's daily activities. The scores are broken down into various components such as task completion, cover sheet utilization, promptness in the application process, offered candidates, prompt submissions, and prompt references.

Candidate Activity Data is collected from the candidate tracking system. It includes various activities candidates perform from the time they enter the system, such as completing the initial employment application, performing job searches, requesting job submissions, reading job update emails, clicking on links in the emails, and logging in to the candidate platform, and more.

Job Submission Fata includes information about each job submission made. It includes the expected weekly hours, the hourly bill rate, and other details about the job posting. It also includes the expected number of weeks worked and contractual start and end dates on each assignment.

Employer Cost data is utilized and relates to employer costs such as FICA, FUTA, SUTA, workers comp, and payroll for the traveling contractor. This data is used in the calculation of Gross Profit/GP Margins.

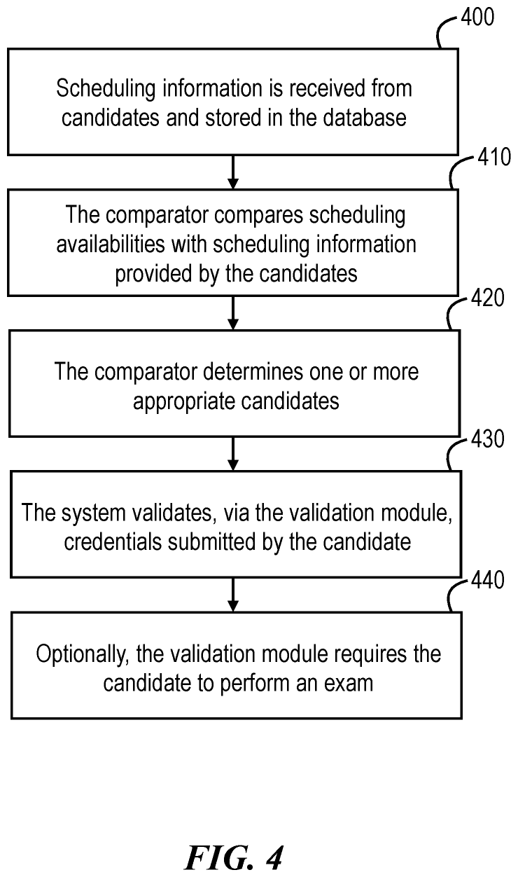

FIG. 4 illustrates a method for obtaining staffing for a plurality of staffing opportunities available at various healthcare facilities. In step 400, scheduling information is received from a plurality of candidates and stored in the database to permit the system to analyze the data as described hereinabove. In step 410, a comparator compares scheduling availabilities with scheduling information provided by the candidates. The comparator may compare preferred date availabilities, time availabilities, locations, and other known parameters to determine one or more appropriate candidates in step 420. In step 430, the system may validate, via the validation module, credentials submitted by the candidate. In some cases, the validation module may require the candidate to perform an exam, submit documents, submit references, or other processes which validate the experience and knowledge of the candidate in optional step 440.

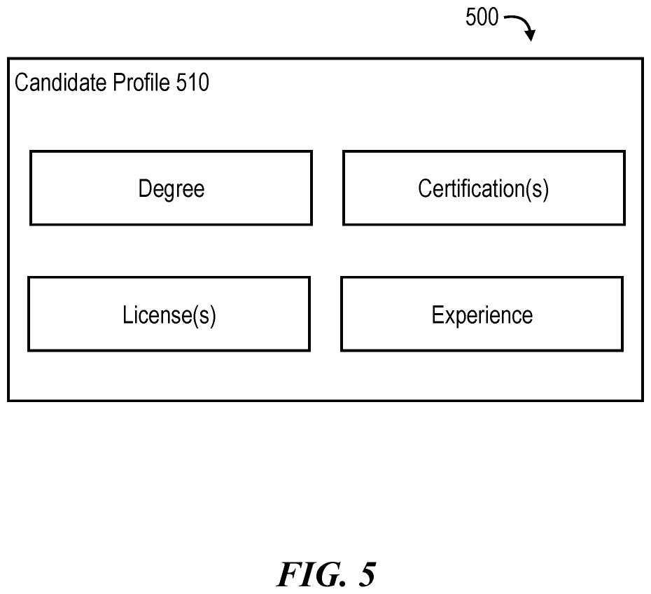

FIG. 5 illustrates schematic of the candidate credential table 500 having data stored in the database and organized by the verification module to display candidates and their associated credentials. The candidate profile 510 comprises data related to the job seeker, such as experience, education, tasks performed, skills possessed, scheduling information, etc.

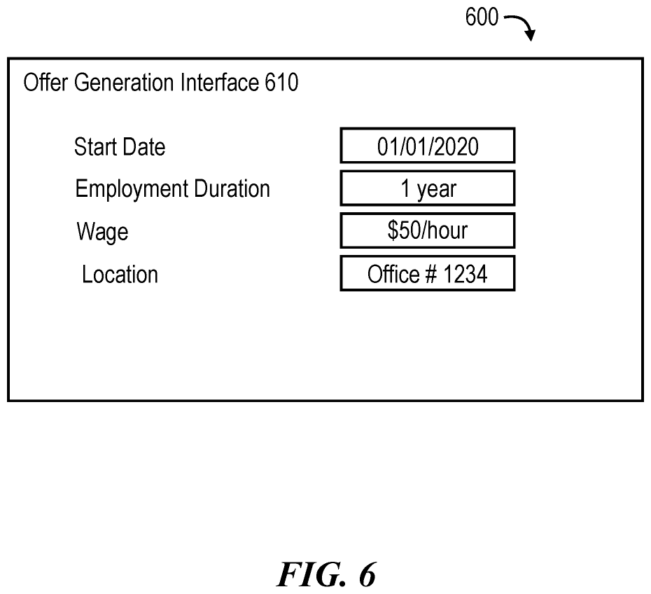

FIG. 6 illustrates an offer generation system 600 having an offer generation interface 610. The offer generation interface 610 permits the system to generate an offer for employments including a plurality of employment terms 620, e.g., start date, duration of employment (if applicable), wages (i.e., salary or hourly rate), and other employment terms known in the arts. The verification module may then verify each credential with documents uploaded to the database.

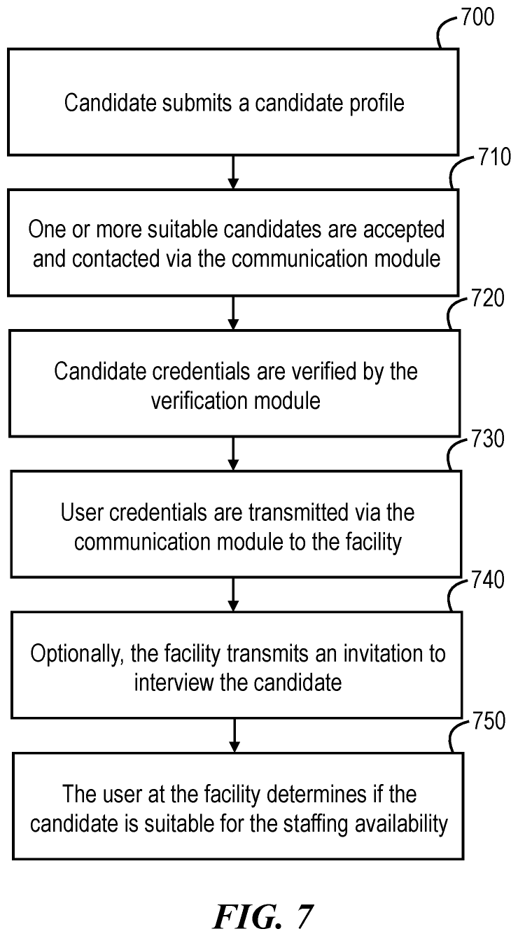

FIG. 7 illustrates a flowchart of a method for staffing a temporary position at a facility. In step 700, the candidate submits a candidate profile (see FIG. 5) which is stored in the database. A user at the facility may search, or otherwise review a plurality of candidate profiles to determine a suitable candidate. In some embodiments, the system may automatically present the user at the facility with candidates who meet predefined parameters. In step 710, one or more suitable candidates are accepted and contacted via the communication module. In step 720, the verification module verifies the candidate's credentials. In step 730, the communication module transmits the user's credentials to the user at the facility and permits the user at the facility to review the user's credentials. In optional step 740, the user at the facility transmits an invitation to interview with one or more person(s) at the facility, which has verified and contacted the candidate. In step 750, the user at the facility determines if the candidate is suitable and selects an accept, deny, and/or a further inquiry option to either accept the candidate for an available position, deny the candidate for an available position, or request another interview, request more information, or request another interaction from the candidate.

The primary goal of the system is to develop a predictive model that can accurately forecast future revenues. Leveraging historical revenue data and other crucial metrics such as recruiter scores and candidate activities, the system aims to identify patterns and trends that will allow it to make accurate revenue predictions. The resulting model will serve as a strategic tool, informing business decisions and helping optimize agency processes.

The model development process will encompass several steps, including feature engineering, model selection, model training and evaluation, as well as model optimization and validation. Each of these steps is crucial to ensure the robustness and reliability of the predictive model.

The model includes time-lagged features. For the WoW revenue data, one approach would be to generate lagged features. This refers to a technique where historical data points are used to predict the next ones. For example, the revenue from 1, 2, or 3 weeks ago could be helpful in predicting future revenue.

Recruiter scores encompass several different aspects, including task completion, Coversheet utilization, prompt application process, offered candidates, prompt submissions, and prompt references. Each of these components may be treated as an individual feature, in addition to the total recruiter score. It may also be beneficial to create new features that represent the interaction between these components and to explore other aspects of the recruiting process that affect future revenues.

Various candidate activities such as completing the initial employment application, completing skills checklists, entering professional references, eliciting responses from their references, performing job searches, subscribing for job update emails, opening the job update emails, and clicking on links in the emails could each be a feature. It may also be helpful to create binary (yes/no) features indicating whether or not certain key activities have taken place.

Many machine learning algorithms perform better when the input numerical variables fall into a similar range. For continuous variables, the system may apply transformations, such as logarithmic or square root transformations, to reduce skewness of the distribution and bring the variables into a similar range.

Interaction features are new features that represent the combination of existing features. For example, if certain types of candidate activities tend to occur together and this combination is particularly predictive of revenue, you could create a new feature that represents this interaction.

Recruiter scores in a staffing agency context can serve as an indicator of recruiter productivity, effectiveness, and the potential revenue they can generate. As such, a significant component of this project is to assess the relationship between recruiter scores and revenue, and then to leverage this information to improve the predictive models.

The system aims to evaluate whether and how well recruiter scores predict future revenues. The process involves building a predictive model with recruiter scores as independent variables and revenues as the dependent variable, allowing us to quantify the impact of recruiter scores on revenues. We'll then determine which components of recruiter scores (task completion, coversheet utilization, prompt application process, offered candidates, prompt submissions, and prompt references) are the most significant predictors.

The system may use various insights from the above processes to improve the existing recruiter scoring system, thereby driving higher performance. If certain components of the recruiter scores have a weak correlation with revenues or don't contribute significantly to the predictive power of the model, the system may revise how scare are calculated. By refining the recruiter scoring system, the system will drive higher productivity, effectiveness, and ultimately, revenue generation.

Given that the response variable (revenue) is continuous, the system begins with a linear regression model as it is simple and interpretable. The system will use recruiter scores as the independent variable and future revenue as the dependent variable. This model will provide a preliminary view of the relationship between these two variables.

With the linear regression model chosen, the system will split the dataset into a training set and a test set. The training set will be used to train the model while the test set will be used to evaluate its performance.

Once the model is trained, the system will use it to predict future revenues in the test set. The predictions will be compared to the actual values to evaluate the model's performance. The most common metrics for this purpose are Root Mean Squared Error (RMSE) and Mean Absolute Error (MAE). The RMSE gives higher weight to larger errors, while the MAE treats all errors equally.

The coefficients of the linear regression model can provide insights into the relationship between the recruiter scores and the future revenues. A positive coefficient suggests that as the recruiter score increases, future revenues also increase. Conversely, a negative coefficient suggests the opposite. The absolute value of the coefficient indicates the strength of this relationship.

Linear regression has certain assumptions such as linearity, independence, homoscedasticity, and normality. The system will check these assumptions using techniques like residual plots and normal probability plots.

Understanding the influence of each feature used in the predictive model is of utmost importance, not just for model interpretability, but also for enhancing the understanding of the recruitment process. This section will address the assessment of feature importance within the model.

For tree-based models, such as Decision Trees, Random Forest, or Gradient Boosting, feature importance can be directly obtained, providing insight into which variables contribute the most to the model's predictive power. However, it is worth noting that this approach is based on the model's internal workings and might not completely reflect the causal relationships in the system.

A tree-based model is trained on the dataset, which includes the recruiter scores and their components as independent variables and future revenue as the dependent variable.

The feature importance is extracted from the model. In tree-based models, the importance of a feature is often determined based on the amount that each attribute split point improves the model's performance, weighted by the number of observations the node is responsible for. The more an attribute is used to make key decisions, the higher its relative importance.

The relative importance score for each feature is assessed. The higher the score, the more important the feature in contributing to the model's prediction. With the scores computed, the system will gain an understanding of which aspects of the recruiter score have the most influence on the predictive model, thereby allowing us to know which features contribute the most to the revenues. It may also highlight areas of the recruiter score that have a negligible effect on revenue, providing an opportunity to revisit the scoring system and potentially refine the metrics.

One point to note is that feature importance does not imply causality. A feature may be important in the context of the model, but it may not be the cause of increased revenue. Therefore, while this analysis will provide valuable insights, it should be used in combination with business understanding and further statistical analysis to draw more reliable conclusions.

Also, the system may want to use permutation importance or SHAP (SHapley Additive explanations) values for a more reliable estimate of feature importance, especially if the dataset has features that are highly correlated. Permutation importance is a measure of how much the model's performance decreases when a feature's information is randomly shuffled, thus breaking the correlation between the feature and the target. SHAP values provide an even deeper understanding by explaining the contribution of each feature to every individual prediction.

In conclusion, the analysis of feature importance will provide us valuable insights into the components of the recruiter score that significantly contribute to the predictive power of the model, guiding us in the process of further optimization.

Recruiter Score Optimization allows for the leveraging the insights and information derived from the analysis and modeling process. The aim is to create a more accurate and reliable system of scoring that can more effectively predict future revenues. The recruiter score is a cumulative calculation of several key performance indicators, including task completion, Coversheet utilization, promptness of the application process, offered candidates, prompt submissions, and prompt references. Through the modeling process, the system identifies the most influential indicators and assessed their impact on the prediction of future revenues.

A major aspect of this optimization process involves revising the scoring components based on their importance. For instance, if task completion has shown to be a crucial determinant of revenue prediction in the model, it might be beneficial to allocate more points to this category, enhancing its impact on the overall score. Moreover, the introduction of additional indicators could also provide value. Indicators that consider the recruiter's interaction and relationship with the candidates, such as response time or satisfaction ratings from candidates, could offer a more comprehensive understanding of the recruiter's effectiveness.

To facilitate a seamless transition to the optimized scoring system, it is also important to ensure that all recruiters are adequately trained to understand the changes. Clear communication of the rationale behind the adjustments, as well as how they can improve their scores and ultimately contribute to the agency's success, will be crucial for buy-in and successful implementation.

Lastly, the system provides ongoing monitoring and regular reviews of the scoring system to ensure its sustained effectiveness and to make further adjustments as necessary. This iterative process will allow the staffing agency to continually optimize the predictive capabilities of the recruiter scores, thereby enhancing its revenue forecasting and strategic planning capabilities.

One of the unique aspects of the system's approach involves incorporating candidate activity data into the staffing process. This represents a significant shift in the standard operational model and positions the agency to better serve both the clients and candidates, ultimately leading to higher revenues. In order to accomplish this, the system may develop a weighted score system for different candidate activities based on their predictive value for revenue, building and evaluating a predictive model using the new weighted score, and providing suggestions for improving the recruiter scores based on the insights gained from model building and evaluation.

Candidate activities tracked from the time they enter the system range from completing initial employment applications to requesting job submissions and performing job searches. It also includes the way they interact with job update emails and the candidate platform.

By carefully assessing and weighting these activities, the system can predict the candidates who are most likely to contribute to revenue generation, allowing us to refine the staffing process and optimize their sources. It is a goal that the prioritized candidates are those most likely to lead to successful placements and satisfied clients, enhancing the competitiveness and boosting the revenue in the competitive travel nurse staffing marketplace.

Candidate activity data provides key insights into the behavior of potential employees and understanding these patterns can enhance the staffing process and improve the predictive revenue model. The candidate activities that the systems monitors spans a wide range, from the completion of initial employment applications and self-assessment of skills to more engaged actions like job searches, requesting submissions, and interacting with curated job update emails.
a comprehensive exploratory data analysis (EDA) on candidate activity data may be performed. The basic descriptive statistics for each candidate activity such as means, medians, standard deviations, and ranges may be calculated. This helps to provide a general understanding of the distribution of each variable. Next, the system examines trends and patterns over time. How does the frequency of each activity change from day to day, or week to week? Are there certain periods when activity spikes or dips? Next, the system assesses the relationship between various candidate activities and generated revenues. This includes looking at how strongly each activity is correlated with revenue, and whether the relationship is positive or negative.

In some embodiments, the system groups similar candidates based on their activity patterns using clustering techniques. This might reveal interesting segments of candidates who engage in similar behaviors, which could be predictive of revenue generation.

In some embodiments, the system compares the activities of high-revenue and low-revenue generating candidates to identify key differentiators. This can help the system understand which activities are most indicative of a candidate's potential value to the agency.

Through this detailed analysis of candidate activity data, the system aim to gain insights that will help us refine the staffing process, enhancing the agency's ability to identify and prioritize high-potential candidates and improve revenue predictions. These findings will also inform the development of a weighted scoring system for candidate activities in the following section.

The integration of candidate activity data into the revenue prediction model requires the development of a weighted score system. In this system, different candidate activities are assigned specific weights based on their predicted impact on future revenues. The aim is to determine which candidate activities contribute most to future revenues and should therefore be prioritized and encouraged.

To begin with, the system analyzes each candidate activity to determine its relevance to revenue generation. This involves examining the correlation of each activity with revenues and analyzing trends and patterns in the data. The system will initially focus on activities like the completion of the initial employment application, skills checklist, references requirements, and job submissions. The system will also consider secondary activities, such as email opens, clicks on job update emails, and login frequencies on the candidate platform.

After identifying the significant activities, the system assigns weights to these activities. The weights are determined based on their correlation with future revenues-activities that have a stronger positive correlation with revenues are assigned higher weights. For instance, candidates who complete their application process quickly and request job submissions to a variety of job offerings may be assigned a higher weight than those who merely open emails.

Using the weights assigned to different activities, an algorithm is developed to calculate the weighted candidate activity score. The score is a sum of each activity score (calculated as the product of the activity's weight and the count of the activity) for a given candidate. This score provides an overall measure of a candidate's potential revenue-generating capacity.

Finally, the weighted candidate activity score is integrated into the predictive model as a new feature. This feature enhances the model's ability to predict future revenues by taking into account the candidate's level of engagement and their likelihood to generate revenue.

In conclusion, the weighted score development stage is critical in tailoring the predictive model to accurately reflect the influence of candidate activity on future revenues. By quantifying and integrating candidate activities into the model, the system can increase the model's predictive accuracy and provide actionable insights to optimize the staffing process.

Given the nature of the data and the problem at hand, the system may use ensemble learning methods such as Gradient Boosting or Random Forests due to their ability to model complex relationships and interactions between variables. For comparison, the system may also train a simple linear regression model and a neural network model to understand if a more complex model is justified.

Incorporating the weighted score of candidate activity into the model will require thoughtful feature engineering. The system may use raw weighted score and derived features like moving averages or rates of change. Before training, features will be appropriately scaled, and categorical features will be one-hot encoded.

The system will split the data into training and validation datasets in order to evaluate model performance during the training process. The system may also use cross-validation techniques to ensure robust evaluation. The model will then be trained on the prepared dataset, with the goal of minimizing a selected loss function that aligns with the business objectives.

Hyperparameters of the model will be fine-tuned to achieve the best possible performance. Techniques such as grid search or random search will be employed to automate the search for optimal hyperparameters.

The model will be evaluated on the basis of various performance metrics including Mean Absolute Error (MAE), Root Mean Squared Error (RMSE), and R-squared value. The system will also assess the model's performance on different segments of the data, to ensure its effectiveness across various scenarios.

To gain a deeper understanding of the model's predictions, the system will conduct a feature importance analysis. This will help us understand which aspects of candidate activity are most influential in predicting future revenues and validate if the weighted scoring system aligns with these findings.

The aim of this section is to create a robust and accurate predictive model that leverages the weighted score for candidate activity to predict future revenues. The insights gleaned from this model will enable us to make informed decisions on how to optimize the staffing process.

The previous sections have detailed the process of assessing the current recruiter score system and its impact on revenue prediction. After careful analysis of the correlation between recruiter scores and revenues, and the evaluation of predictive models based on these scores, the system can now suggest ways to improve the effectiveness of recruiter scores.

The goal is to ensure that the recruiter score provides an accurate reflection of the recruiter's efficiency and potential to generate revenue.

Each component of the recruiter score holds a certain level of importance when it comes to predicting revenues. Based on the feature importance analysis, the system can identify which components of the score have the most impact on the revenue prediction. For instance, if ‘CoversheetUtilization’ or ‘promptSubmissions’ have a higher importance, it implies that the recruiters' using the Coversheet Generator and submit candidates quickly significantly affects revenue generation. On the other hand, components like ‘promptReferences’ or ‘taskCompletion’ might have less impact, implying that these activities are less crucial for revenue prediction.

Once the system identifies the key components affecting revenues, the system can optimize the score calculation. For instance, if ‘promptApplicationProcess’ and ‘offeredCandidates’ are crucial for revenue generation, the system may consider increasing the maximum points for these components. This could incentivize recruiters to focus more on these areas, thereby potentially increasing revenues.

This section outlines the process of developing a predictive system for revenue calculations. This system will act as a crucial tool in predicting future revenues based on expected contract duration, billable hours, and hourly bill rate for each candidate. The idea is to construct an accurate prediction model that estimates the expected duration of contracts and uses this information to forecast potential revenues.

The proposed model will integrate various features such as historical contracts, submissions, billable hours, and hourly bill rate to predict expected contract duration for each candidate. This holistic approach takes into account a variety of factors to ensure accurate prediction and better decision-making processes.

However, building this model requires a series of steps, each having its importance and role in the overall construction of the predictive system. These steps include data preparation, model building, validation, and evaluation, followed by the integration of this model into the existing system to predict future revenues based on the predicted contract duration.

Each of these steps will be discussed in detail in the subsequent sections. They will guide the software development team through the process, providing clear instructions and considerations to ensure the successful development of the predictive system. By following these steps, the team can work towards building a comprehensive system that not only predicts future revenues but also helps in optimizing the staffing process.

In this disclosure, the various embodiments are described with reference to the flowchart illustrations and/or block diagrams of methods, apparatus (systems), and computer program products. Those skilled in the art would understand that each block of the flowchart illustrations and/or block diagrams, and combinations of blocks in the flowchart illustrations and/or block diagrams, can be implemented by computer readable program instructions. The computer readable program instructions can be provided to a processor of a general purpose computer, special purpose computer, or other programmable data processing apparatus to produce a machine, such that the instructions, which execute via the processor of the computer or other programmable data processing apparatus, create means for implementing the functions or acts specified in the flowchart and/or block diagram block or blocks. The computer readable program instructions can be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks. The computer readable program instructions can be loaded onto a computer, other programmable data processing apparatus, or other device to cause a series of operational acts to be performed on the computer, other programmable apparatus, or other device to produce a computer implemented process, such that the instructions that execute on the computer, other programmable apparatus, or other device implement the functions or acts specified in the flowchart and/or block diagram block or blocks.

In this disclosure, the block diagrams in the Figures illustrate the architecture, functionality, and operation of possible implementations of systems, methods, and computer program products according to the various embodiments. Each block in the flowchart or block diagrams can represent a module, segment, or portion of instructions, which comprises one or more executable instructions for implementing the specified logical function(s). In some embodiments, the functions noted in the blocks can occur out of the order noted in the Figures. For example, two blocks shown in succession can, in fact, be executed concurrently or substantially concurrently, or the blocks can sometimes be executed in the reverse order, depending upon the functionality involved. In some embodiments, each block of the block diagrams and/or flowchart illustration, and combinations of blocks in the block diagrams and/or flowchart illustration, can be implemented by a special purpose hardware-based system that performs the specified functions or acts or carry out combinations of special purpose hardware and computer instructions.

In this disclosure, the subject matter has been described in the general context of computer-executable instructions of a computer program product running on a computer or computers, and those skilled in the art would recognize that this disclosure can be implemented in combination with other program modules. Generally, program modules include routines, programs, components, data structures, etc. that perform particular tasks and/or implement particular abstract data types. Those skilled in the art would appreciate that the computer-implemented methods disclosed herein can be practiced with other computer system configurations, including single-processor or multiprocessor computer systems, mini-computing devices, mainframe computers, as well as computers, hand-held computing devices (e.g., PDA, phone), microprocessor-based or programmable consumer or industrial electronics, and the like. The illustrated embodiments can be practiced in distributed computing environments where tasks are performed by remote processing devices that are linked through a communications network. Some embodiments of this disclosure can be practiced on a stand-alone computer. In a distributed computing environment, program modules can be located in both local and remote memory storage devices.

In this disclosure, the terms “component,” “system,” “platform,” “interface,” and the like, can refer to and/or include a computer-related entity or an entity related to an operational machine with one or more specific functionalities. The disclosed entities can be hardware, a combination of hardware and software, software, or software in execution. For example, a component can be a process running on a processor, a processor, an object, an executable, a thread of execution, a program, and/or a computer. By way of illustration, both an application running on a server and the server can be a component. One or more components can reside within a process and/or thread of execution and a component can be localized on one computer and/or distributed between two or more computers. In another example, respective components can execute from various computer readable media having various data structures stored thereon. The components can communicate via local and/or remote processes such as in accordance with a signal having one or more data packets (e.g., data from one component interacting with another component in a local system, distributed system, and/or across a network such as the Internet with other systems via the signal). As another example, a component can be an apparatus with specific functionality provided by mechanical parts operated by electric or electronic circuitry, which is operated by a software or firmware application executed by a processor. In such a case, the processor can be internal or external to the apparatus and can execute at least a part of the software or firmware application. As another example, a component can be an apparatus that provides specific functionality through electronic components without mechanical parts, wherein the electronic components can include a processor or other means to execute software or firmware that confers at least in part the functionality of the electronic components. In some embodiments, a component can emulate an electronic component via a virtual machine, e.g., within a cloud computing system.

The phrase “application” as is used herein means software other than the operating system, such as Word processors, database managers, Internet browsers and the like. Each application generally has its own user interface, which allows a user to interact with a particular program. The user interface for most operating systems and applications is a graphical user interface (GUI), which uses graphical screen elements, such as windows (which are used to separate the screen into distinct work areas), icons (which are small images that represent computer resources, such as files), pull-down menus (which give a user a list of options), scroll bars (which allow a user to move up and down a window) and buttons (which can be “pushed” with a click of a mouse). A wide variety of applications is known to those in the art.

The phrases “Application Program Interface” and API as are used herein mean a set of commands, functions and/or protocols that computer programmers can use when building software for a specific operating system. The API allows programmers to use predefined functions to interact with an operating system, instead of writing them from scratch. Common computer operating systems, including Windows, Unix, and the Mac OS, usually provide an API for programmers. An API is also used by hardware devices that run software programs. The API generally makes a programmer's job easier, and it also benefits the end user since it generally ensures that all programs using the same API will have a similar user interface.

The phrase “central processing unit” as is used herein means a computer hardware component that executes individual commands of a computer software program. It reads program instructions from a main or secondary memory, and then executes the instructions one at a time until the program ends. During execution, the program may display information to an output device such as a monitor.

The term “execute” as is used herein in connection with a computer, console, server system or the like means to run, use, operate or carry out an instruction, code, software, program and/or the like.

In this disclosure, the descriptions of the various embodiments have been presented for purposes of illustration and are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein. Thus, the appended claims should be construed broadly, to include other variants and embodiments, which may be made by those skilled in the art.

What is claimed is:

1. A system for obtaining staffing for a plurality of staffing opportunities available at various healthcare facilities, the system comprising:
at least one user computing device in operable connection with a user network;
an application server in operable communication with the user network, the application server configured to host an application program for a staffing system at a healthcare facility.
a calendar module to receive scheduling information via the computing device, the scheduling information corresponding to a plurality of staffing availabilities provided by one or more healthcare professionals;
a hiring system configured to transmit the plurality of staffing availabilities to the plurality of candidates;
a comparator to compare data pertaining to the plurality of candidates with the plurality of staffing availabilities to match one or more of the plurality of candidates with one or more of the plurality of staffing availabilities;
a revenue prediction module to provide revenue forecasting related to each of the plurality of candidates; and
an evaluation module to receive a plurality of recruiter data and to generate a recruiter score using the plurality of data, wherein the revenue prediction module and the evaluation module provide a predictive model for revenue calculations.

2. The system of claim 1, further comprising an evaluation module configured to receive one or more evaluations and correspond the evaluation to one of the plurality of healthcare professionals.

3. The system of claim 1, further comprising a plurality of candidate activity, the plurality of candidate activity data transmitted to the revenue prediction module to identify revenue-generating candidates.

4. The system of claim 1, wherein the evaluation module provides a recruiter score optimization to optimize future revenues.

5. The system of claim 1, wherein one or more revenue calculation predictions are provided based on the plurality of candidate activity data.

6. A system for obtaining staffing for a plurality of staffing opportunities available at various healthcare facilities, the system comprising:
a computing device in operable communication with a database to store information related to a plurality of candidates and a plurality of healthcare facilities;
a calendar module to receive scheduling information via the computing device, the scheduling information corresponding to a plurality of staffing availabilities provided by one or more healthcare professionals;
a hiring system configured to transmit the plurality of staffing availabilities to the plurality of candidates;
a comparator to compare data pertaining to the plurality of candidates with the plurality of staffing availabilities to match one or more of the plurality of candidates with one or more of the plurality of staffing availabilities
a verification module to verify one or more credentials provided by the plurality of candidates; and
a revenue prediction module to provide revenue forecasting, the revenue prediction module to receive a plurality of candidate activity to aid in the identification of revenue-generating candidates; and
an evaluation module to receive a plurality of recruiter data and to generate a recruiter score using the plurality of data, wherein the revenue prediction module and the evaluation module provide a predictive model for revenue calculations.

7. The system of claim 6, further comprising an evaluation module configured to receive one or more evaluations and correspond the evaluation to one of the plurality of healthcare professionals.

8. The system of claim 7, further comprising a plurality of candidate activity, the plurality of candidate activity data transmitted to the revenue prediction module to identify revenue-generating candidates.

9. The system of claim 8, wherein the evaluation module provides a recruiter score optimization to optimize future revenues.

10. The system of claim 9, wherein one or more revenue calculation predictions are provided based on the plurality of candidate activity data.

11. The system of claim 10, wherein the one or more credentials are comprised of one or more of the following: a vaccination history, one or more licenses, and one or more qualifications.

12. The system of claim 11, further comprising an employment history stored in the database, wherein the employment history viewable via the computing device.

13. The system of claim 12, further comprising an evaluation module configured to receive one or more evaluations and correspond the evaluation to one of the plurality of healthcare professionals.

14. The system of claim 13, wherein the one or more evaluations are displayed on the computing device.

15. The system of claim 14, further comprising a calendar module to indicate one or more employable candidates.

16. The system of claim 15, wherein the scheduling information comprises one or more of the following: one or more dates available, one or more days of the week available, one or more locations, a travel distance, and one or more times available.

17. The system of claim 16, further comprising a communication module to provide a communication means between the plurality of healthcare facilities and the plurality of candidates.

18. A method for obtaining staffing for a plurality of staffing opportunities available at various healthcare facilities, the method comprising the steps of:
receiving a plurality of scheduling information from a plurality of candidates;
storing the plurality of staffing availabilities in a database in operable communication with a computing device;
comparing, via a comparator in operable communication with the database via a server, the plurality of staffing availabilities with scheduling information provided by the plurality of candidates;
determining, via a scheduling module, appropriate healthcare professionals to correspond to the plurality of scheduling information to determine one or more employable candidates;
forecasting, via a revenue prediction module, a revenue associated with each of the one or more employable candidate using a plurality of candidate activity data;
wherein the forecasting of revenue is performed using historical contracts, submissions, billable hours, and hourly bill rates.
